(19) United States
(12) Patent Application Publication
(10) Pub. No.: US 2022/0409079 A1
(43) Pub. Date: Dec. 29, 2022

(54) Heart Rate Estimation Method and Apparatus, and Electronic Device Applying Same

(71) Applicant: ARCSOFT CORPORATION LIMITED, Hangzhou, Zhejiang (CN)

(72) Inventors: Zhirui ZHANG, Hangzhou, Zhejiang (CN); Tianjiao XU, Hangzhou, Zhejiang (CN); Jin WANG, Hangzhou, Zhejiang (CN)

(21) Appl. No.: 17/779,152

(22) PCT Filed: Nov. 18, 2020
(86) PCT No.: PCT/CN2020/129836

§ 371 Date: May 24, 2022

(30) Foreign Application Priority Data
Nov. 25, 2019 (CN) 201911166001.2

(51) Int. Cl. A61B 5/024; A61B 5/00

(52) U.S. Cl. CPC A61B 5/02433; A61B 5/7257; A61B 5/7203

(57) ABSTRACT

The present invention discloses a heart rate estimation method and apparatus, and an electronic device applying same. The heart rate estimation method includes: acquiring a face video; performing face detection on the face video to extract a local face area that is set as heart rate estimation; performing first processing on values of pixel points in the local face area to obtain an initial heart rate signal; performing time-frequency domain conversion on the initial heart rate signal to obtain a frequency domain signal; and performing second processing on the frequency domain signal within a heart rate estimation range to obtain a heart rate estimation value.

### CROSS-REFERENCE TO RELATED APPLICATION

The present application claims the priority of Chinese Application No. 201911166001.2, filed in the Chinese Patent Office on Nov. 25, 2019, and entitled “Heart Rate Estimation Method and Apparatus, and Electronic Device Applying Same”, the entire contents of which are herein incorporated by reference.

### TECHNICAL FIELD

The present invention relates to a computer vision technology, especially to a heart rate estimation method and apparatus, and an electronic device applying same.

### BACKGROUND

An electrocardiogram is a technique that uses an electrocardiograph to record, from a body surface, a pattern of point activity changes produced by a heart within each cardiac cycle. Although this method has high accuracy, the instrument is expensive and requires professional operation, such that the equipment is cumbersome, and the use scenarios are extremely limited.

Photo plethyamo graphy (PPG) is a non-invasive measurement method for monitoring blood volume changes in living body tissues by means of photoelectric means. When light of a certain wavelength is irradiated on a skin surface such as between fingers, the light will be captured by a photoelectric receiver in a transmission or reflection manner. During the whole process, the light is weakened due to the absorption by skin, muscles, blood and the like, and thus the light intensity reaching the photoelectric receiver will be reduced. The weakening effect of the skin, the muscles and the like on the light intensity is constant, while the weakening effect of blood in blood vessels on the light will show a pulsatile change with the beating of the heart. When the heart contracts, the blood volume in the blood vessels increases, the absorbed light intensity increases, and the light intensity received by the photoelectric receiver decreases accordingly. When the heart dilates, the condition is opposite. By converting the light intensity into an electrical signal, the change in the volume pulse blood flow can be obtained. This method requires a close contact between a sensor and a fixed human body part, thereby having many restrictions on the use methods of users and use scenarios.

### SUMMARY

Embodiments of the present disclosure provide a heart rate estimation method and apparatus, and an electronic device applying same, so as to at least solve the technical problem in the prior art that heart rate estimation can only be realized in a contact manner.

According to one aspect of the embodiments of the present invention, a heart rate estimation method is provided, including: acquiring a face video; performing face detection on the face video to extract a local face area that is set as heart rate estimation; performing first processing on values of pixel points in the local face area to obtain an initial heart rate signal; performing time-frequency domain conversion on the initial heart rate signal to obtain a frequency domain signal; and performing second processing on the frequency domain signal within a heart rate estimation range to obtain a heart rate estimation value.

Optionally, the face video is collected by using an infrared camera.

Optionally, the local face area includes an area below the eyes.

Optionally, the heart rate estimation method further includes: combining face key point positioning with the face detection to extract the local face area that is set as heart rate estimation.

Optionally, the step of performing first processing on the values of the pixel points in the local face area to obtain the initial heart rate signal includes: performing weighted average on the values of the pixel points in the local face area according to weights, and taking the value of weighted average as a luminance signal of the current frame; and constituting the initial heart rate signal by the luminance signal of the current frame and luminance signals of previous N historical frames.

Optionally, the step of performing time-frequency domain conversion on the initial heart rate signal to obtain the frequency domain signal includes: converting the initial heart rate signal from a time domain into a frequency domain by using fast Fourier transform, to obtain the frequency domain signal.

Optionally, the weight is set according to the position of the pixel point in the local face area, and the closer the pixel point is to an edge position in the local face area, the smaller the weight corresponding to the pixel point is.

Optionally, before performing the time-frequency domain conversion on the initial heart rate signal, the heart rate estimation method can further include: performing first denoising processing on the initial heart rate signal in the time domain, wherein the first denoising processing method includes at least one of the following: S-G (Savitzky-Golay) filtering, detrend (Detrend) filtering, moving average filtering, normalization (Normalize) processing, and bandpass (Bandpass) filtering.

Optionally, the step of performing second processing on the frequency domain signal within the heart rate estimation range to obtain the heart rate estimation value includes: performing peak value detection on the frequency domain signal within the heart rate estimation range to obtain peak values; sorting the peak values to obtain a sorting result; calculating a confidence coefficient according to the sorting result; and obtaining the heart rate estimation value according to the confidence coefficient.

Optionally, the step of performing second processing on the frequency domain signal within the heart rate estimation range to obtain the heart rate estimation value includes: performing peak value detection on the frequency domain signal within the heart rate estimation range to obtain a highest peak value; taking the frequency of the highest peak value as main frequency, and calculating energy of a first-order harmonic and a second-order harmonic corresponding to the main frequency, to obtain an energy calculation value; dividing the energy calculation value by energy of the remaining frequency other than the main frequency, to obtain a confidence coefficient; and obtaining the heart rate estimation value according to the confidence coefficient.

Optionally, the heart rate estimation range is preset.

Optionally, the heart rate estimation method further includes: judging whether a detection time exceeds a second threshold value; and when the detection time exceeds the second threshold value, performing third processing on the frequency domain signal to obtain the heart rate estimation range.

Optionally, the step of performing third processing on the frequency domain signal to obtain the heart rate estimation range includes: continuously selecting consecutive X frames or X seconds of frequency domain signals through a sliding window to obtain a heart rate value, buffering the heart rate value, acquiring the heart rate estimation range by using a deep learning method, repeating the action within M frames or M seconds, and averaging all acquired heart rate estimation ranges to obtain a final heart rate estimation range.

Optionally, the peak values are sorted in a traversal manner in a descending order of the peak values, a highest peak value and a second peak value are selected as the sorting result, and a ratio of the highest peak value to the second peak value is taken as the confidence coefficient.

Optionally, the step of obtaining the heart rate estimation value according to the confidence coefficient includes: comparing the confidence coefficient with a first threshold value to obtain a comparison result; when the confidence coefficient is less than the first threshold value, the comparison result indicates that the frequency domain signal is seriously polluted by noise, then discarding the current result, and detecting the next frame; and when the confidence coefficient is not less than the first threshold value, the comparison result indicates that the frequency domain signal is not polluted by noise or the noise pollution is relatively small, then acquiring the frequency corresponding to the highest peak value as the heart rate estimation value.

Optionally, before performing second processing on the frequency domain signal, the heart rate estimation method further includes: performing second denoising processing on the frequency domain signal, wherein the second denoising processing method includes at least one or more of the following: discrete Fourier transform (DFT), and bandpass (Bandpass) filtering.

According to another aspect of the embodiments of the present invention, a heart rate estimation apparatus is further provided, including: a camera shooting unit, configured to acquire a face video; a detection unit, configured to perform face detection on the face video to extract a local face area that is set as heart rate estimation; a first processing unit, configured to perform first processing on values of pixel points in the local face area to obtain an initial heart rate signal; a conversion unit, configured to perform time-frequency domain conversion on the initial heart rate signal to obtain a frequency domain signal; and a second processing unit, configured to perform second processing on the frequency domain signal within a heart rate estimation range to obtain a heart rate estimation value.

According to another aspect of the embodiments of the present invention, a storage medium is further provided, including a stored program, wherein the program, when running, controls a device where the storage medium is located to execute the heart rate estimation method in any of above embodiments.

Optionally, the second processing unit includes: a peak detection module, configured to perform peak value detection on the frequency domain signal within the heart rate estimation range to obtain peak values; a sorting module, configured to sort the peak values to obtain a sorting result; a confidence coefficient calculation module, configured to obtain a confidence coefficient according to the sorting result; and an estimation module, configured to obtain the heart rate estimation value according to the confidence coefficient.

Optionally, the second processing unit includes: a highest peak detection module, configured to perform peak value detection on the frequency domain signal within the heart rate estimation range to obtain a highest peak value; an energy calculation module, configured to take the frequency of the highest peak value as main frequency, and calculate energy of a first-order harmonic and a second-order harmonic corresponding to the main frequency, to obtain an energy calculation value;

a confidence coefficient calculation module, configured to divide the energy calculation value by energy of the remaining frequency other than the main frequency, to obtain a confidence coefficient; and an estimation module, configured to obtain the heart rate estimation value according to the confidence coefficient.

Optionally, the heart rate estimation apparatus further includes: a judging module, configured to judge whether a detection time exceeds a second threshold value; and a third processing module configured to, when the detection time exceeds the second threshold value, perform third processing on the frequency domain signal to obtain the heart rate estimation range.

According to another aspect of the embodiments of the present invention, an electronic device is further provided, including: a processor; and a memory, configured to store executable instructions of the processor, wherein the processor is configured to execute the heart rate estimation method in any of above embodiments by executing the executable instructions.

In the embodiments of the present invention, the following steps are executed: acquiring the face video; performing face detection on the face video to extract the local face area that is set as heart rate estimation; performing first processing on the values of the pixel points in the local face area to obtain the initial heart rate signal; performing time-frequency domain conversion on the initial heart rate signal to obtain the frequency domain signal; and performing second processing on the frequency domain signal within the heart rate estimation range to obtain the heart rate estimation value. Therefore, the technical problem in the related art is solved that heart rate estimation can only be realized in a contact manner.

### BRIEF DESCRIPTION OF THE DRAWINGS

The drawings described herein are used for providing a further understanding of the present invention and constitute a part of the present application. Exemplary embodiments of the present invention and descriptions thereof are used for explaining the present invention, but do not constitute improper limitations of the present invention. In the drawings:

FIG. 1 is a flowchart of a first optional heart rate estimation method according to an embodiment of the present invention;

FIG. 2 is a schematic diagram of an optional extracted local face area according to an embodiment of the present invention;

FIG. 3 is a flowchart of a second optional heart rate estimation method according to an embodiment of the present invention;

FIG. 4 is an application scenario diagram of a heart rate estimation method provided according to an embodiment of the present invention; and

FIG. 5 is a structural block diagram of an optional heart rate estimation apparatus according to an embodiment of the present invention.

### DETAILED DESCRIPTION OF THE EMBODIMENTS

In order that those skilled in the art can better understand the solutions of the present invention, a clear and complete description of technical solutions in the embodiments of the present invention will be given below, in combination with the drawings in the embodiments of the present invention. Apparently, the embodiments described below are merely a part, but not all, of the embodiments of the present invention. All of other embodiments, obtained by those of ordinary skill in the art based on the embodiments of the present invention without any creative effort, fall into the protection scope of the present invention.

It should be illustrated that, the terms “first” and “second” and the like in the specification, claims and the above-mentioned drawings of the present invention are used for distinguishing similar objects, and are not necessarily used for describing a specific sequence or precedence order. It should be understood that the sequences used in this way can be interchanged under appropriate circumstances, so that the embodiments of the present invention described herein can be implemented in a sequence other than those illustrated or described herein. Furthermore, the terms “including” and “having”, and any variations thereof are intended to cover non-exclusive inclusions, for example, processes, methods, systems, products or devices including a series of steps or units are not necessarily limited to those clearly listed steps or units, but can include other steps or units that are not clearly listed or are inherent to these processes, methods, products or devices.

The embodiments of the present invention can be applied to an electronic device with at least one camera shooting unit, and the electronic device can include: smart phones, tablet computers, electronic readers, desktop computers, workstations, servers, personal digital assistants (PDAs), portable multimedia players (PMP), medical devices, cameras or wearable devices (accessories such as watches, bracelets, glasses and headsets), electronic clothing, skin chips that can be implanted in vivo, vehicle-mounted electronic instruments, etc.

The flowchart of an optional heart rate estimation method according to an embodiment of the present invention will be described below. It should be illustrated that, the steps shown in the flowchart of the drawings can be executed in a computer system, such as a group of computer-executable instructions. Moreover, although a logical sequence is shown in the flowchart, in some cases, the steps shown or described can be executed in a sequence different from that herein.

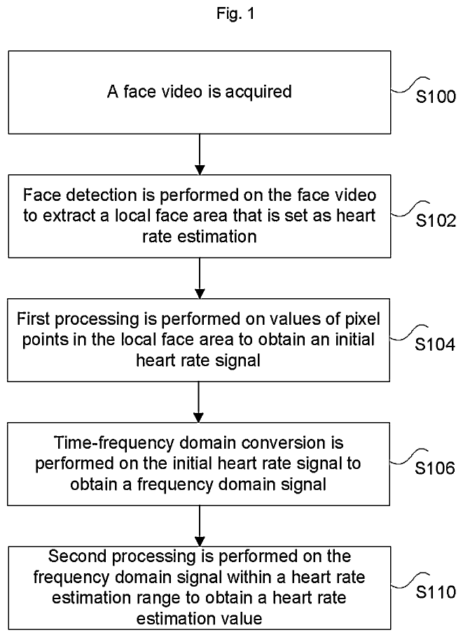

Referring to FIG. 1, it is a flowchart of a first optional heart rate estimation method according to an embodiment of the present invention. As shown in FIG. 1, the heart rate estimation method includes the following steps:

S100: acquiring a face video.

In an optional embodiment, in the present step, the face video is acquired by using a camera shooting apparatus (e.g., an RGB camera, an infrared camera, and so on), and the frame rate is usually greater than 25 fps, so as to keep the succession of a picture, which is conducive to capturing a heart rate state. When an infrared camera is utilized, since infrared light is almost invisible to naked eyes, no influence is generated on human activities; and moreover, the use of the infrared camera can avoid the influence of dark light and complex light, thus having certain anti-interference and anti-blocking capabilities. In addition, because of exact transmitting and receiving wave bands of an infrared light source, a large amount of noise that cannot be avoided due to the use of ordinary RGB camera solutions can be effectively removed, and active light supplementation is realized. The infrared light source can be a built-in light source of the infrared camera, and can also be an external independent light source. The camera shooting apparatus can be an independent camera, or integrated with other cameras in whole or in part to form one or more camera shooting modules, and can be installed independently or installed on the electronic device in an embedded or external manner.

S102: performing face detection on the face video to extract a local face area that is set as heart rate estimation.

In an optional embodiment, in the present step, the local face area that is set as heart rate estimation can also be extracted by combining face key point positioning with the face detection. By introducing face key points, it is possible to extract the local face area that is set as heart rate estimation more accurately, so that in the case of face shaking, a heart rate estimation value will not be deviated due to motion blur and facial deformation.

In an optional embodiment, the local face area extracted in the present step includes an area below the eyes. Since the area below the eyes has more veins distributed and is not easily blocked compared with other places on the face, by extracting the area below the eyes as the local face area, the accuracy of the heart rate estimation method can be improved.

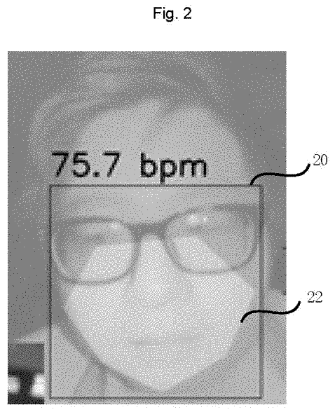

For example, as shown in FIG. 2, it is a schematic diagram of an optional extracted local face area according to an embodiment of the present invention, the local face area that is set as heart rate estimation is extracted by combining face key point positioning with the face detection. In FIG. 2, a rectangular frame 20 represents a face area determined by face detection, and an irregular mask frame 22 represents the local face area extracted by the face key points.

Of course, those skilled in the art can be aware that, other areas on the face, such as a forehead and a cheek, can also be extracted as the local face area; or, without creative effort, those skilled in the art can also extract other human body areas with rich veins as detection areas for heart rate estimation, such as a neck and a wrist.

S104: performing first processing on values of pixel points in the local face area to obtain an initial heart rate signal.

In an optional embodiment, the present step includes: performing weighted average on the values of the pixel points in the local face area according to weights, and taking the value of weighted average as a luminance signal of the current frame; and constituting the initial heart rate signal by the luminance signal of the current frame and luminance signals of previous N historical frames, and N being an integer greater than or equal to 1, wherein the weight can be set according to the position of the pixel point in the local face area, and the closer the pixel point is to an edge position in the local face area, the smaller the weight corresponding to the pixel point is. The value range of the weight can be 0-1.

S106: performing time-frequency domain conversion on the initial heart rate signal to obtain a frequency domain signal.

In an optional embodiment, the step of performing time-frequency domain conversion on the initial heart rate signal to obtain the frequency domain signal includes: converting the initial heart rate signal from a time domain into a frequency domain by using fast Fourier transform, to obtain the frequency domain signal.

In an optional embodiment, before performing the time-frequency domain conversion on the initial heart rate signal, the heart rate estimation method can further include: performing first denoising processing on the initial heart rate signal in the time domain, wherein the first denoising processing method includes at least one of the following: S-G (Savitzky-Golay) filtering, detrend (Detrend) filtering, moving average filtering, normalization (Normalize) processing, and bandpass (Bandpass) filtering. The S-G (Savitzky-Golay) filtering and detrend (Detrend) filtering can be configured to reduce the influence of signal baseline translation on the signal (for example, to eliminate the influence of ambient light). The moving average filtering can be configured to remove random noise from the signal. The normalization (Normalize) processing can facilitate signal processing and improve the computational efficiency. By means of the bandpass (Bandpass) filtering, the signal in a normal heart rate frequency domain band can be processed. By means of the first denoising processing, high-frequency noise, low-frequency noise, and noise introduced by other factors (for example, motion) can be removed, so that the heart rate signal is clearer, and the periodicity is more prominent.

S110: performing second processing on the frequency domain signal within a heart rate estimation range to obtain a heart rate estimation value.

In an optional embodiment, the step of performing second processing on the frequency domain signal to obtain the heart rate estimation value includes:

S1100: performing peak value detection on the frequency domain signal within the heart rate estimation range to obtain peak values;

S1102: sorting the peak values to obtain a sorting result;

S1104: obtaining a confidence coefficient according to the sorting result; and

S1106: obtaining the heart rate estimation value according to the confidence coefficient.

In an optional embodiment, the peak values are sorted in a traversal manner in a descending order of the peak values, and the sorting result is acquired as needed, for example, a highest peak value and a second peak value are selected as the sorting result. A ratio of the highest peak value to the second peak value is taken as the confidence coefficient, that is, confidence coefficient=highest peak value/second peak value.

In another optional embodiment, the step of performing second processing on the frequency domain signal to obtain the heart rate estimation value includes:

S1110: performing peak value detection on the frequency domain signal within the heart rate estimation range to obtain a highest peak value;

S1112: taking the frequency of the highest peak value as main frequency, and calculating energy of a first-order harmonic and a second-order harmonic corresponding to the main frequency, to obtain an energy calculation value;

S1114: dividing the energy calculation value by energy of the remaining frequency other than the main frequency, so as to obtain a confidence coefficient; and

S1116: obtaining the heart rate estimation value according to the confidence coefficient.

In an optional embodiment, the heart rate estimation range can be preset, for example, the heart rate estimation range can be preset to 50 beats/min to 180 beats/min according to a heart rate limit value of a human body.

In an optional embodiment, the step of obtaining the heart rate estimation value according to the confidence coefficient includes: comparing the confidence coefficient with a first threshold value to obtain a comparison result; when the confidence coefficient is less than the first threshold value, the comparison result indicates that the frequency domain signal is seriously polluted by noise, then discarding the current frequency domain signal, and detecting the next frame; and when the confidence coefficient is not less than the first threshold value, the comparison result indicates that the frequency domain signal is not polluted by noise or the noise pollution is relatively small, then acquiring the frequency corresponding to the highest peak value as the heart rate estimation value.

In an optional embodiment, before performing second processing on the frequency domain signal, the heart rate estimation method can further include: performing second denoising processing on the frequency domain signal, wherein the second denoising processing method includes at least one or more of the following: discrete Fourier transform (DFT), and bandpass (Bandpass) filtering. By means of the bandpass (Bandpass) filtering, the signal in the normal heart rate frequency domain band can be processed. By performing the second denoising processing on the frequency domain signal, a required principal component signal can be further highlighted.

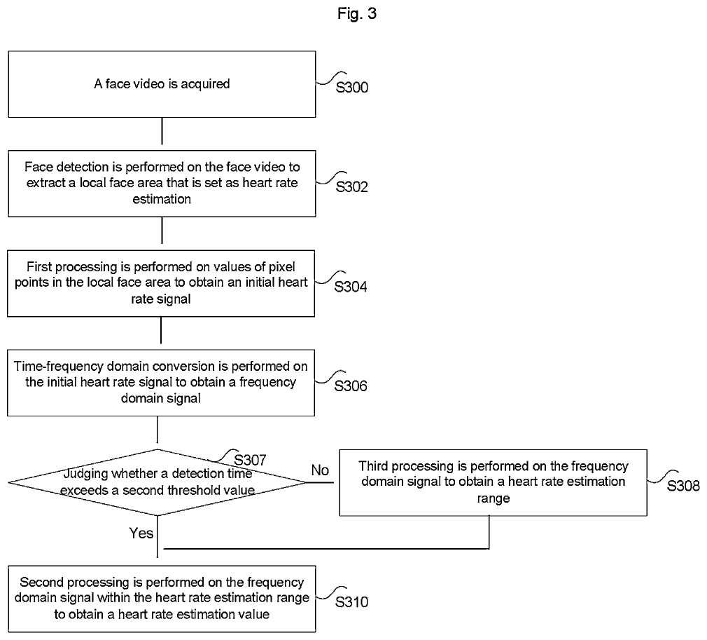

In the heart rate estimation method provided by the above embodiment, if the heart rate estimation range is preset, there may be a case where an output value is unstable due to inappropriate range setting. In order to achieve more accurate and more stable heart rate estimation, the present invention further provides another heart rate estimation method with an automatic range estimation function. Referring to FIG. 3, it is a flowchart of a second optional heart rate estimation method according to an embodiment of the present invention. As shown in FIG. 3, the heart rate estimation method includes the following steps:

S300: acquiring a face video;

S302: performing face detection on the face video to extract a local face area that is set as heart rate estimation;

S304: performing first processing on values of pixel points in the local face area to obtain an initial heart rate signal;

S306: performing time-frequency domain conversion on the initial heart rate signal to obtain a frequency domain signal; and

S307: judging whether a detection time exceeds a second threshold value;

S308: performing third processing on the frequency domain signal to obtain a heart rate estimation range; and

S310: performing second processing on the frequency domain signal within the heart rate estimation range to obtain a heart rate estimation value.

In the heart rate estimation method provided by the present embodiment, the steps S300, S302, S304, S306 and S310 respectively correspond to the steps S100, S102, S104, S106 and S110 in the first embodiment, and thus will not be repeated herein. The difference is that the heart rate estimation method provided by the present embodiment further includes the step S307 and the step S308, which will be described in detail below.

In the step S307, the second threshold value can be set to be M frames or M seconds, M is an integer greater than or equal to 1, the detection time is timed, and a timing value is compared with the second threshold value to judge whether the detection time exceeds the second threshold value; and if the detection time exceeds the second threshold value, the step S307 is skipped to the step S308, and third processing is performed on the frequency domain signal to obtain the heart rate estimation range. The step of performing third processing on the frequency domain signal to obtain the heart rate estimation range can include: continuously selecting consecutive X frames or X seconds of frequency domain signals through a sliding window to obtain a heart rate value, buffering the heart rate value, acquiring the heart rate estimation range by using a deep learning method, repeating the action within M frames or M seconds, and averaging all acquired heart rate estimation ranges to obtain a final heart rate estimation range. If the detection time does not exceed the second threshold value, the step is skipped to the step S310 to perform second processing on the frequency domain signal within the heart rate estimation range, so as to obtain the heart rate estimation value.

According to the heart rate estimation method provided by the embodiment of the present invention, in addition to detecting heart rate estimation, the method can also be used for acquiring cardiovascular parameters such as a respiratory rate, a blood oxygen content (SpO2) and a blood pressure, so as to monitor physical conditions of a person. This heart rate estimation method can be applied to various mobile platforms, vehicle-mounted chips, embedded chips and the like, and it requires no large and complex hardware device, so that the detection process is simple and fast, and no contact and no harm is brought to the human body. At the same time, the method has sufficient accuracy, and solves the problem of the traditional contact detection method of relying on complex hardware device and requiring contact with the human body, therefore the robustness and application range of the heart rate estimation method are greatly improved.

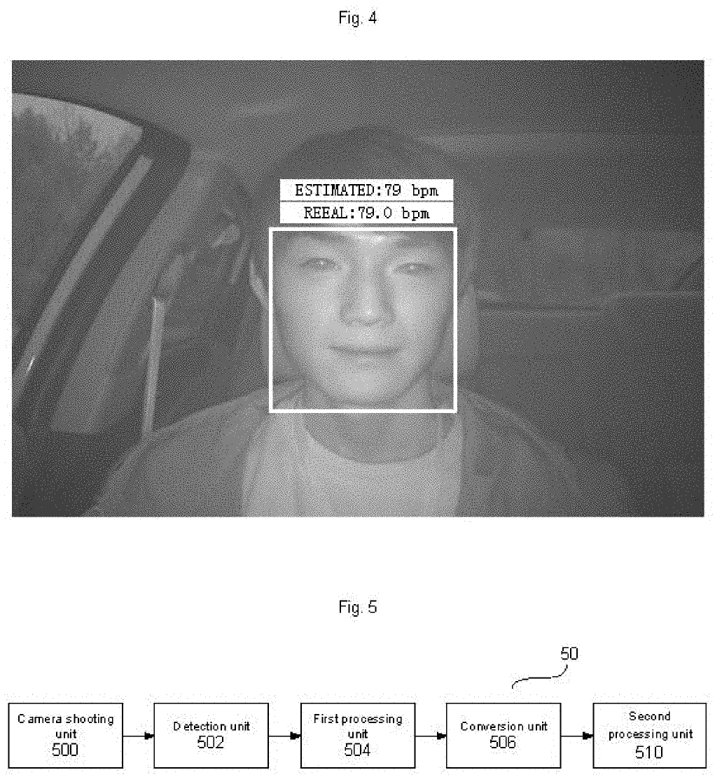

In an application scenario of the embodiment of the present invention, as shown in FIG. 4, it is an application scenario diagram of a heart rate estimation method provided according to an embodiment of the present invention. If the heart rate estimation method is used for monitoring the physical conditions of a driver, the problem can be avoided that a traditional contact heat rate device needs to be worn on the driver, thus generating certain influence on driving. It can be seen from detection results shown in FIG. 4 that, the similarity accuracy of an estimated heart rate value (Estimated) obtained by using the heart rate estimation method and a real heart rate value (Real) is very high. In addition, accurate heart rate estimation can still be realized under dark light conditions such as at nighttime, tunnels and backlight, so as to continuously track and judge the physical conditions of the driver, when it is monitored that the heart rate of the driver is abnormal, an alarm prompt can be given, and an assisted driving function is started. Of course, those skilled in the art can be aware that, the heart rate estimation method provided according to the embodiment of the present invention can also be applied to other scenarios, such as sleep heart rate monitoring.

According to another aspect of the embodiments of the present invention, a heart rate estimation apparatus is further provided. Referring to FIG. 5, it is a structural block diagram of an optional heart rate estimation apparatus according to an embodiment of the present invention. As shown in FIG. 5, the heart rate estimation apparatus 50 includes a camera shooting unit 500, a detection unit 502, a first processing unit 504, a conversion unit 506 and a second processing unit 510.

Each unit included in the heart rate estimation apparatus 50 will be described in detail below.

The camera shooting unit 500 is configured to acquire a face video.

In an optional embodiment, the camera shooting unit 500 can be an RGB camera, an infrared camera, and so on, and the frame rate is usually greater than 25 fps, so as to keep the succession of a picture, which is conducive to capturing a heart rate state. When an infrared camera is utilized, since infrared light is almost invisible to naked eyes, no influence is generated on human activities; and moreover, the use of the infrared camera can avoid the influence of dark light and complex light, thus having certain anti-interference and anti-blocking capabilities. In addition, because of exact transmitting and receiving wave bands of an infrared light source, a large amount of noise that cannot be avoided due to the use of ordinary RGB camera solutions can be effectively removed, and active light supplementation is realized. The infrared light source can be a built-in light source of the infrared camera, and can also be an external independent light source. The camera shooting unit 500 can be an independent camera, or integrated with other cameras in whole or in part to form one or more camera shooting modules, and can be installed independently or installed on an electronic device in an embedded or external manner.

The detection unit 502 is configured to perform face detection on the face video to extract a local face area that is set as heart rate estimation.

In an optional embodiment, in addition to including a face detection module 5020 that is configured to perform face detection, the detection unit 502 can further include a face key point positioning module 5022, which is configured to be combined with the face detection module 5020 to extract the local face area that is set as heart rate estimation. By introducing the face key point positioning module 5022, it is possible to extract the local face area that is set as heart rate estimation more accurately, so that in the case of face shaking, a heart rate estimation value will not be deviated due to motion blur and facial deformation.

In an optional embodiment, the local face area extracted in the present step includes an area below the eyes. Since the area below the eyes has more veins distributed and is not easily blocked compared with other places on the face, by extracting the area below the eyes as the local face area, the accuracy of the heart rate estimation method can be improved.

The first processing unit 504 is configured to perform first processing on values of pixel points in the local face area to obtain an initial heart rate signal.

In an optional embodiment, the first processing unit 504 includes a luminance signal acquisition module 5040, configured to perform weighted average on the values of the pixel points in the local face area according to weights, and take the value of weighted average as a luminance signal of the current frame; and an initial heart rate signal acquisition module 5042, configured to acquire the initial heart rate signal according to the luminance signal of the current frame and luminance signals of previous N historical frames, and N being an integer greater than or equal to 1, wherein the weight can be set according to the position of the pixel point in the local face area, and the closer the pixel point is to an edge position in the local face area, the smaller the weight corresponding to the pixel point is. The value range of the weight can be 0-1.

The conversion unit 506 is configured to perform time-frequency domain conversion on the initial heart rate signal to obtain a frequency domain signal.

In an optional embodiment, the conversion unit 506 can convert the initial heart rate signal from a time domain into a frequency domain by using fast Fourier transform, to obtain the frequency domain signal.

In an optional embodiment, the heart rate estimation apparatus 50 can further include: a first denoising module configured to, before performing time-frequency domain conversion on the initial heart rate signal, perform first denoising processing on the initial heart rate signal in the time domain, wherein the first denoising processing method includes at least one of the following: S-G (Savitzky-Golay) filtering, detrend (Detrend) filtering, moving average filtering, normalization (Normalize) processing, and bandpass (Bandpass) filtering. The S-G (Savitzky-Golay) filtering and detrend (Detrend) filtering can be configured to reduce the influence of signal baseline translation on the signal (for example, to eliminate the influence of ambient light). The moving average filtering can be configured to remove random noise from the signal. The normalization (Normalize) processing can facilitate signal processing and improve the computational efficiency. By means of the bandpass (Bandpass) filtering, the signal in a normal heart rate frequency domain band can be processed. By means of the first denoising processing, high-frequency noise, low-frequency noise, and noise introduced by other factors (for example, motion) can be removed, so that the heart rate signal is clearer, and the periodicity is more prominent.

The second processing unit 510 is configured to perform second processing on the frequency domain signal within a heart rate estimation range to obtain a heart rate estimation value.

In an optional embodiment, the second processing unit 510 includes:

a peak detection module 5100, configured to perform peak value detection on the frequency domain signal within the heart rate estimation range to obtain peak values;

a sorting module 5102, configured to sort the peak values to obtain a sorting result;

a confidence coefficient calculation module 5104, configured to calculate a confidence coefficient according to the sorting result; and an estimation module 5106, configured to obtain the heart rate estimation value according to the confidence coefficient.

In an optional embodiment, the peak values are sorted in a traversal manner in a descending order of the peak values, and the sorting result is acquired as needed, for example, a highest peak value and a second peak value are selected as the sorting result. A ratio of the highest peak value to the second peak value is taken as the confidence coefficient, that is, confidence coefficient=highest peak value/second peak value.

In an optional embodiment, the second processing unit 510 includes:

a highest peak detection module 5110, configured to perform peak value detection on the frequency domain signal within the heart rate estimation range to obtain a highest peak value;

an energy calculation module 5112, configured to take the frequency of the highest peak value as main frequency, and calculate energy of a first-order harmonic and a second-order harmonic corresponding to the main frequency, to obtain an energy calculation value;

a confidence coefficient calculation module 5114, configured to divide the energy calculation value by energy of the remaining frequency other than the main frequency, to obtain a confidence coefficient; and

an estimation module 5116, configured to obtain the heart rate estimation value according to the confidence coefficient.

In an optional embodiment, the heart rate estimation range can be preset, for example, the heart rate estimation range can be preset to 50 beats/min to 240 beats/min according to a heart rate limit value of a human body.

In an optional embodiment, the estimation module 5106 is configured to compare the confidence coefficient with a first threshold value to obtain a comparison result; when the confidence coefficient is less than the first threshold value, the comparison result indicates that the frequency domain signal is seriously polluted by noise, then discard the current frequency domain signal, and detect the next frame; and when the confidence coefficient is not less than the first threshold value, the comparison result indicates that the frequency domain signal is not polluted by noise or the noise pollution is relatively small, then acquire the frequency corresponding to the highest peak value as the heart rate estimation value.

In an optional embodiment, the heart rate estimation apparatus 50 can further include a second denoising module configured to, before performing second processing on the frequency domain signal, perform second denoising processing on the frequency domain signal, wherein the second denoising processing method includes at least one or more of the following: discrete Fourier transform (DFT), and bandpass (Bandpass) filtering. By means of the bandpass (Bandpass) filtering, the signal in the normal heart rate frequency domain band can be processed. By performing the second denoising processing on the frequency domain signal, a required principal component signal can be further highlighted.

In another optional embodiment, the heart rate estimation apparatus 50 can further include a judging module 507, configured to judge whether a detection time exceeds a second threshold value, wherein the second threshold value can be set to be M frames or M seconds, M is an integer greater than or equal to 1, the detection time is timed, and a timing value is compared with the second threshold value to judge whether the detection time exceeds the second threshold value; and a third processing module 508 configured to, when the detection time exceeds the second threshold value, perform third processing on the frequency domain signal to obtain the heart rate estimation range, wherein the step of performing third processing on the frequency domain signal to obtain the heart rate estimation range can include: continuously selecting consecutive X frames or X seconds of frequency domain signals through a sliding window to obtain a heart rate value, buffering the heart rate value, acquiring the heart rate estimation range by using a deep learning method, repeating the action within M frames or M seconds, and averaging all acquired heart rate estimation ranges to obtain a final heart rate estimation range. If the detection time does not exceed the second threshold value, the second processing unit 510 performs second processing on the frequency domain signal within the heart rate estimation range to obtain the heart rate estimation value.

According to another aspect of the embodiments of the present invention, an electronic device is further provided, including: a processor; and a memory, configured to store executable instructions of the processor, wherein the processor is configured to execute the heart rate estimation method in any of above embodiments by executing the executable instructions.

According to another aspect of the embodiments of the present invention, a storage medium is further provided, including a stored program, wherein the program, when running, controls a device where the storage medium is located to execute the heart rate estimation method in any of above embodiments.

The serial numbers of the above embodiments of the present invention are only for description, and do not represent the advantages or disadvantages of the embodiments.

In the above embodiments of the present invention, the description of each embodiment has its own emphasis. For parts that are not described in detail in a certain embodiment, reference can be made to related descriptions of other embodiments.

In the several embodiments provided by the present application, it should be understood that, the disclosed technical content can be implemented in other manners. The apparatus embodiments described above are merely exemplary, for example, the division of the units is only a logic function division, there can be other division manners in practical implementation, for example, a plurality of units or components can be combined or integrated to another system, or some features can be omitted or not implemented. From another point of view, the displayed or discussed mutual coupling or direct coupling or communication connection can be indirect coupling or communication connection of units or modules through some interfaces, and can be in electrical, mechanical or other forms.

The units described as separate components can be separated physically or not, components displayed as units can be physical units or not, namely, can be located in one place, or can be distributed on a plurality of units. A part of or all of the units can be selected to implement the purposes of the solutions in the present embodiment according to actual demands.

In addition, the functional units in various embodiments of the present invention can be integrated in a processing unit, or the units individually exist physically, or two or more units are integrated in one unit. The integrated unit can be implemented in the form of hardware, and can also be implemented in the form of a software functional unit.

If the integrated unit is implemented in the form of the software functional unit and is sold or used as an independent product, it can be stored in a computer-readable storage medium. Based on this understanding, the technical solutions of the present invention substantially, or the part contributing to the prior art, or part of or all the technical solutions can be implemented in the form of a software product, the computer software product is stored in a storage medium, and includes several instructions for enabling a computer device (which can be a personnel computer, a server, or a network device or the like) to execute all or part of the steps of the method in various embodiments of the present invention. The foregoing storage medium includes a variety of media capable of storing program codes, such as a USB disk, a read-only memory (ROM, Read-Only Memory), a random access memory (RAM, Random Access Memory), a mobile hard disk, a magnetic disk, or an optical disk.

The foregoing descriptions are merely specific embodiments of the present invention. It should be pointed out that, those of ordinary skill in the art can make several improvements and modifications without departing from the principles of the present invention, and these improvements and modifications should also be regarded as the protection scope of the present invention.

### INDUSTRIAL APPLICABILITY

The solutions provided by the embodiments of the present application can monitor the physical conditions of a person. The technical solutions provided by the embodiments of the present application can be applied to an electronic device with at least one camera shooting unit, for example, applicable to various mobile platforms, vehicle-mounted chips, embedded chips and the like, and it requires no large and complex hardware device, so that the detection process is simple and fast, and no contact and no harm is brought to the human body. At the same time, the accuracy is enough, the problem of a traditional contact detection method of relying on complex hardware device and requiring contact with the human body is solved, and the robustness and application range of the heart rate estimation method are greatly improved. The physical conditions of a driver are continuously tracked and judged, when it is monitored that the heart rate of the driver is abnormal, an alarm prompt can be given, and an assisted driving function is started.

1. A heart rate estimation method, comprising:
acquiring a face video;
performing face detection on the face video to extract a local face area that is set as heart rate estimation;
performing first processing on values of pixel points in the local face area to obtain an initial heart rate signal;
performing time-frequency domain conversion on the initial heart rate signal to obtain a frequency domain signal; and
performing second processing on the frequency domain signal within a heart rate estimation range to obtain a heart rate estimation value.

2. The heart rate estimation method as claimed in claim 1, wherein the face video is collected by using an infrared camera.

3. The heart rate estimation method as claimed in claim 1, wherein the local face area comprises an area below the eyes.

4. The heart rate estimation method as claimed in claim 1, further comprising: combining face key point positioning with the face detection to extract the local face area that is set as heart rate estimation.

5. The heart rate estimation method as claimed in claim 1, wherein the step of performing first processing on the values of the pixel points in the local face area to obtain the initial heart rate signal comprises: performing weighted average on the values of the pixel points in the local face area according to weights, and taking the value of weighted average as a luminance signal of the current frame; and constituting the initial heart rate signal by the luminance signal of the current frame and luminance signals of previous N historical frames.

6. The heart rate estimation method as claimed in claim 1, wherein the step of performing time-frequency domain conversion on the initial heart rate signal to obtain the frequency domain signal comprises: converting the initial heart rate signal from a time domain into a frequency domain by using fast Fourier transform, to obtain the frequency domain signal.

7. The heart rate estimation method as claimed in claim 5, wherein the weight is set according to the position of the pixel point in the local face area, and the closer the pixel point is to an edge position in the local face area, the smaller the weight corresponding to the pixel point is.

8. The heart rate estimation method as claimed in claim 1, wherein before performing the time-frequency domain conversion on the initial heart rate signal, the heart rate estimation method further comprises: performing first denoising processing on the initial heart rate signal in the time domain, wherein the first denoising processing method includes at least one of the following: Savitzky-Golay filtering, detrend filtering, moving average filtering, normalization processing, and bandpass filtering.

9. The heart rate estimation method as claimed in claim 1, wherein the step of performing second processing on the frequency domain signal within the heart rate estimation range to obtain the heart rate estimation value comprises:
performing peak value detection on the frequency domain signal within the heart rate estimation range to obtain peak values;
sorting the peak values to obtain a sorting result;
obtaining a confidence coefficient according to the sorting result; and
obtaining the heart rate estimation value according to the confidence coefficient.

10. The heart rate estimation method as claimed in claim 1, wherein the step of performing second processing on the frequency domain signal within the heart rate estimation range to obtain the heart rate estimation value comprises:
performing peak value detection on the frequency domain signal within the heart rate estimation range to obtain a highest peak value;
taking the frequency of the highest peak value as main frequency, and calculating energy of a first-order harmonic and a second-order harmonic corresponding to the main frequency, to obtain an energy calculation value;
dividing the energy calculation value by energy of the remaining frequency other than the main frequency, to obtain a confidence coefficient; and
obtaining the heart rate estimation value according to the confidence coefficient.

11. The heart rate estimation method as claimed in claim 1, wherein the heart rate estimation range is preset.

12. The heart rate estimation method as claimed in claim 10, wherein the heart rate estimation method further comprises:
judging whether a detection time exceeds a second threshold value; and
when the detection time exceeds the second threshold value, performing third processing on the frequency domain signal to obtain the heart rate estimation range.

13. The heart rate estimation method as claimed in claim 12, wherein the step of performing third processing on the frequency domain signal to obtain the heart rate estimation range comprises: continuously selecting consecutive X frames or X seconds of frequency domain signals through a sliding window to obtain a heart rate value, buffering the heart rate value, acquiring the heart rate estimation range by using a deep learning method, repeating action within M frames or M seconds, and averaging all acquired heart rate estimation ranges to obtain a final heart rate estimation range, wherein the action comprises the steps of continuously selecting consecutive X frames or X seconds of frequency domain signals through a sliding window to obtain a heart rate value, buffering the heart rate value, acquiring the heart rate estimation range by using a deep learning method.

14. The heart rate estimation method as claimed in claim 9, wherein the peak values are sorted in a traversal manner in a descending order of the peak values, a highest peak value and a second peak value are selected as the sorting result, and a ratio of the highest peak value to the second peak value is taken as the confidence coefficient.

15. The heart rate estimation method as claimed in claim 12, wherein the step of obtaining the heart rate estimation value according to the confidence coefficient comprises:
comparing the confidence coefficient with a first threshold value to obtain a comparison result;
when the confidence coefficient is less than the first threshold value, the comparison result indicates that the frequency domain signal is seriously polluted by noise, then discarding the current result, and detecting the next frame; and
when the confidence coefficient is not less than the first threshold value, the comparison result indicates that the frequency domain signal is not polluted by noise or the noise pollution is relatively small, then acquiring the frequency corresponding to the highest peak value as the heart rate estimation value.

16. The heart rate estimation method as claimed in claim 1, wherein before performing second processing on the frequency domain signal, the heart rate estimation method further comprises: performing second denoising processing on the frequency domain signal, wherein the second denoising processing method comprises at least one or more of the following: discrete Fourier transform, and bandpass filtering.

17. A heart rate estimation apparatus, comprising:
a camera shooting unit, configured to acquire a face video;
a detection unit, configured to perform face detection on the face video to extract a local face area that is set as heart rate estimation;
a first processing unit, configured to perform first processing on values of pixel points in the local face area to obtain an initial heart rate signal;
a conversion unit, configured to perform time-frequency domain conversion on the initial heart rate signal to obtain a frequency domain signal; and
a second processing unit, configured to perform second processing on the frequency domain signal within a heart rate estimation range to obtain a heart rate estimation value.

18. The heart rate estimation apparatus as claimed in claim 17, wherein the second processing unit comprises:
a peak detection module, configured to perform peak value detection on the frequency domain signal within the heart rate estimation range to obtain peak values;
a sorting module, configured to sort the peak values to obtain a sorting result;
a confidence coefficient calculation module, configured to obtain a confidence coefficient according to the sorting result; and
an estimation module, configured to obtain the heart rate estimation value according to the confidence coefficient.

19. (canceled)

20. (canceled)

21. A storage medium, wherein the storage medium comprises a stored program, and the program, when running, controls a device where the storage medium is located to execute the heart rate estimation method as claimed in claim 1.

22. An electronic device, comprising:
a processor; and
a memory, configured to store executable instructions of the processor,
wherein the processor is configured to execute the heart rate estimation method as claimed in claim 1 by executing the executable instructions.
